(19) United States
(12) Patent Application Publication
(10) Pub. No.: US 2024/0317731 A1
(43) Pub. Date: Sep. 26, 2024

(54) AMINOTHIAZOLE COMPOUNDS AS C-KIT INHIBITORS

(71) Applicant: Theseus Pharmaceuticals, Inc., Cambridge, MA (US)

(72) Inventors: David C. DALGARNO, Cambridge, MA (US); Wei-Sheng HUANG, Cambridge, MA (US); William C. SHAKESPEARE, Cambridge, MA (US)

(73) Assignee: Theseus Pharmaceuticals, Inc., Cambridge, MA (US)

(21) Appl. No.: 18/259,903

(22) PCT Filed: Jan. 5, 2022
(86) PCT No.: PCT/US2022/011306

§ 371 Date: Jun. 29, 2023

Related U.S. Application Data
(60) Provisional application No. 63/134,469, filed on Jan. 6, 2021.

(51) Int. Cl. C07D 417/14; A61K 31/4439

(52) U.S. Cl. CPC C07D 417/14; A61K 31/4439

(57) ABSTRACT

The invention relates to c-Kit inhibitors useful in the treatment of cancers, and other serine-threonine kinase mediated diseases, having a structure according to Formula (I): wherein X¹, R¹, R², m, and n are described herein.

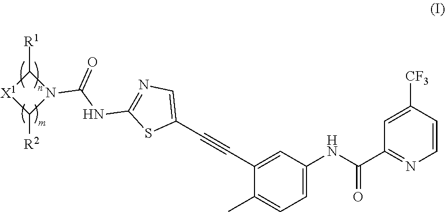

### CROSS-REFERENCE TO RELATED APPLICATIONS

The present application claims benefit of U.S. Provisional Application No. 63/134,469, filed Jan. 6, 2021, which is hereby incorporated by reference in its entirety.

### FIELD OF INVENTION

The present invention is directed to inhibitors of tyrosine-protein kinase Kit (c-Kit) useful in the treatment of diseases or disorders associated with c-Kit. Specifically, the invention is concerned with compounds and compositions inhibiting c-Kit, methods of treating diseases or disorders associated with c-Kit, and methods of synthesis of these compounds.

### BACKGROUND OF THE INVENTION

The discovery that the tyrosine kinase inhibitor (TKI) imatinib inhibits KIT, and its introduction as a treatment, transformed the clinical management of gastrointestinal stromal tumors (GIST) (Corless, C. L. et al., Nat. Rev. Cancer 2011, 11: 865-78). Nonetheless, most imatinib-treated patients ultimately relapse due to outgrowth of clones with secondary, drug-resistant KIT mutations (Heinrich, M. C., et al., J. Clin. Oncol. 2006, 24: 4764-74). Secondary mutations typically occur in the ATP binding pocket encoded by exons 13 and 14, and the activation loop (A-loop) encoded by exons 17 and 18. The challenge of treating imatinib resistant GISTs is compounded by mutational heterogeneity, as patients can harbor multiple different secondary mutations in distinct tumor lesions, or even within different regions of the same lesion (Wardelmann E., et al., Clin. Cancer Res. 2006, 12: 1743-9).

GIST patients with imatinib-resistant tumors are treated with sunitinib, which potently inhibits KIT ATP-pocket mutants (Heinrich, M. C., et al., J Clin Oncol 2008: 26: 5352-9). However, sunitinib is ineffective against A-loop mutants, which account for 50% of imatinib-resistance mutations. This may explain why overall response rates (ORR) are low (7%) and median progression-free survival (PFS) is short (6.2 months; Demetri, G. D., et al., Lancet 2006: 368: 1329-38). Regorafenib was recently approved as third line therapy, but also shows only moderate activity, with ORR of 4.5% and median PFS of 4.8 months (Demetri, G. D., et al., Lancet 2013; 381: 295-302). The KIT inhibitory properties of regorafenib have not yet been analyzed extensively, but both clinical and initial preclinical data suggest a limited spectrum of sensitive KIT mutants (George, S., et al., J. Clin. Oncol. 2012, 30: 2401-7; and Serrano-Garcia, C., et al., ASCO Meeting Abstracts 2013; 31(15_suppl): 10510). Thus, additional agents are needed to overcome resistance mutations in KIT, in particular those in the A-loop.

The KIT inhibitors imatinib, sunitinib and regorafenib are effective GIST therapies, though most patients develop resistance to these drugs due to somatic acquisition of polyclonal secondary KIT mutants. The lack of efficacy of any single agent against the complete set of potential ATP-binding pocket and A-loop secondary mutants makes achievement of prolonged complete disease control in late stage patients challenging. To address this unmet medical need, presented herein are compounds that target a broad range of primary and secondary KIT mutants, including those within the A-loop.

### SUMMARY OF THE INVENTION

The present disclosure provides novel aminothiazole compounds and pharmaceutically acceptable salts as effective c-Kit inhibitors. In particular, compounds described herein can have unexpectedly beneficial pharmacokinetic and therapeutic properties.

A first aspect of the invention relates to compounds of Formula (I):

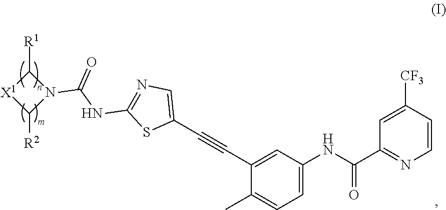

and pharmaceutically acceptable salts, hydrates, solvates, prodrugs, stereoisomers, and tautomers thereof, wherein
n and m are independently 1, 2, or 3;
X¹ is O, NR³, CR^{x}R³ or CHR³;
each R¹, R², and R³ is independently H or X²:
X² is OH, O(C₁-C₆ alkyl), NH₂, NHR⁴, or NR⁴R⁵;
R⁴ and R⁵ are independently C₁-C₆ alkyl or R⁴ and R⁵, when both are present, combine to form a 4- to 6-membered heterocyclyl:
R^{x} is C₁-C₆ alkyl; and
wherein at least one of R¹, R², and R³ is X².

In embodiments, X¹ is O, NR³, or CHR³.

In embodiments, X¹ is CR^{x}R³ and R^{x} is Me.

In embodiments, a compound of Formula (I) has a structure according to Formula (I′),

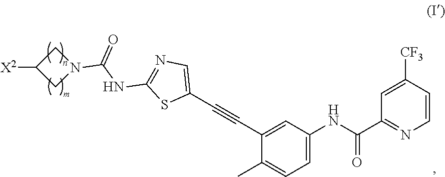

or a pharmaceutically acceptable salt, solvate, prodrug, stereoisomer, or tautomer thereof.

In embodiments, a compound of Formula (I) or (I′) has a structure according to Formula (I′-A),

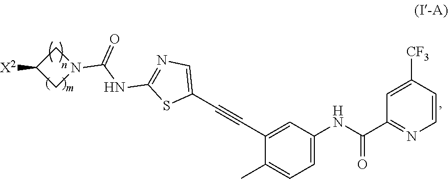

or a pharmaceutically acceptable salt, solvate, prodrug, or tautomer thereof.

In embodiments, a compound of Formula (I) or (I′) has a structure according to Formula (I′-B),

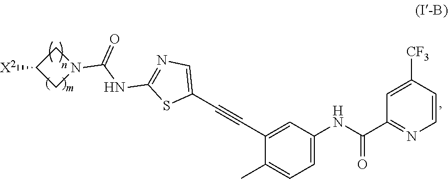

or a pharmaceutically acceptable salt, solvate, prodrug, or tautomer thereof.

In embodiments, X² is OH, NH₂, NHCH₃, or N(CH₃)₂.

In embodiments, X² is NH₂, NHCH₃, or N(CH₃)₂.

In embodiments, a compound of Formula (I) or (I′) is selected from the group consisting of:

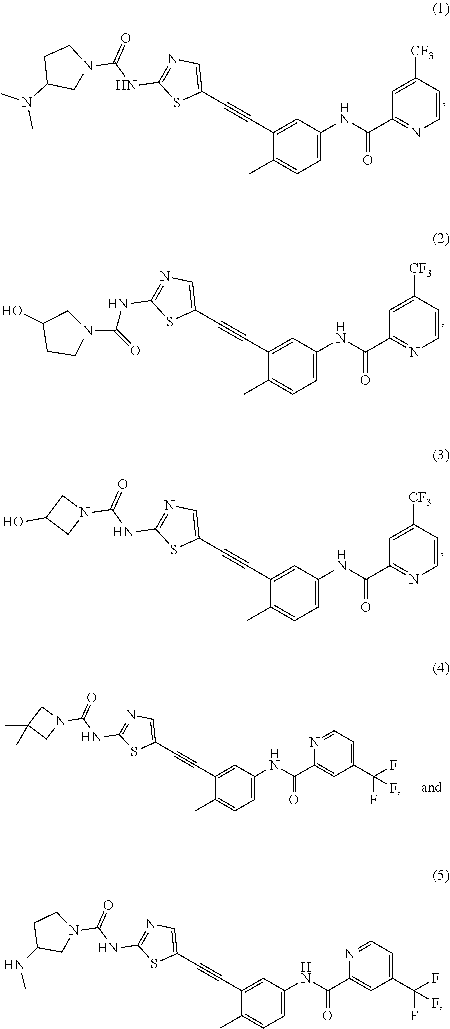 [SMILES: Cc1ccc(NC(=O)c2cc(C(F)(F)F)ccn2)cc1C#Cc1cnc(NC(=O)N2CCC(N(C)C)C2)s1 ; Cc1ccc(NC(=O)c2cc(C(F)(F)F)ccn2)cc1C#Cc1cnc(NC(=O)N2CCC(O)C2)s1 ; Cc1ccc(NC(=O)c2cc(C(F)(F)F)ccn2)cc1C#Cc1cnc(NC(=O)N2CC(O)C2)s1 ; Cc1ccc(NC(=O)c2cc(C(F)(F)F)ccn2)cc1C#Cc1cnc(NC(=O)N2CC(C)(C)C2)s1 ; CNC1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, stereoisomer, or tautomer thereof. In embodiments, a compound of Formula (I) or (I′) is Compound (1),

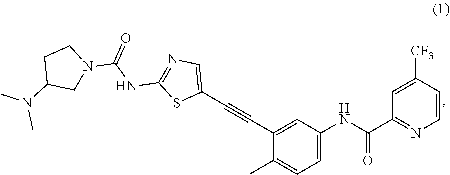 [SMILES: Cc1ccc(NC(=O)c2cc(C(F)(F)F)ccn2)cc1C#Cc1cnc(NC(=O)N2CCC(N(C)C)C2)s1]

or a pharmaceutically acceptable salt, solvate, prodrug, stereoisomer, or tautomer thereof.

In embodiments, a compound of Formula (I) or (I′) is Compound (1a),

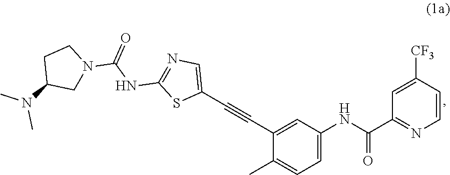 [SMILES: Cc1ccc(NC(=O)c2cc(C(F)(F)F)ccn2)cc1C#Cc1cnc(NC(=O)N2CC[C@H](N(C)C)C2)s1]

or a pharmaceutically acceptable salt, solvate, prodrug, or tautomer thereof.

In embodiments, a compound of Formula (I) or (I′) is Compound (1b),

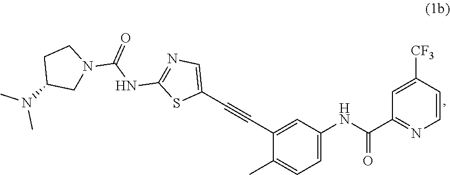 [SMILES: Cc1ccc(NC(=O)c2cc(C(F)(F)F)ccn2)cc1C#Cc1cnc(NC(=O)N2CC[C@@H](N(C)C)C2)s1]

or a pharmaceutically acceptable salt, solvate, prodrug, or tautomer thereof.

In embodiments, a compound of Formula (I) or (I′) is Compound (2),

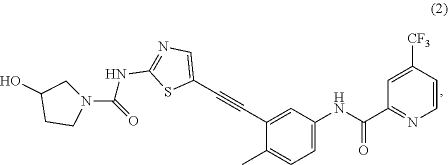 [SMILES: Cc1ccc(NC(=O)c2cc(C(F)(F)F)ccn2)cc1C#Cc1cnc(NC(=O)N2CCC(O)C2)s1]

or a pharmaceutically acceptable salt, solvate, prodrug, stereoisomer, or tautomer thereof.

In embodiments, a compound of Formula (I) or (I′) is Compound (2a),

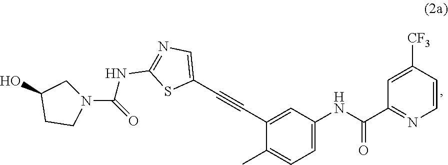 [SMILES: Cc1ccc(NC(=O)c2cc(C(F)(F)F)ccn2)cc1C#Cc1cnc(NC(=O)N2CC[C@@H](O)C2)s1]

or a pharmaceutically acceptable salt, solvate, prodrug, or tautomer thereof.

In embodiments, a compound of Formula (I) or (I′) is Compound (2b),

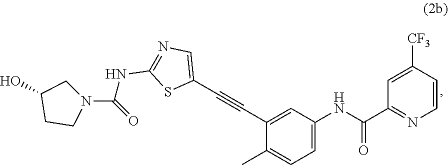 [SMILES: Cc1ccc(NC(=O)c2cc(C(F)(F)F)ccn2)cc1C#Cc1cnc(NC(=O)N2CC[C@H](O)C2)s1]

or a pharmaceutically acceptable salt, solvate, prodrug, or tautomer thereof.

In embodiments, a compound of Formula (I) or (I′) is Compound (3),

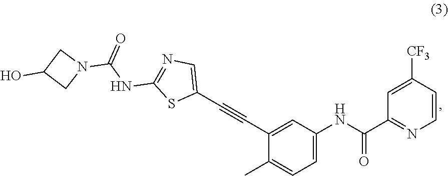 [SMILES: Cc1ccc(NC(=O)c2cc(C(F)(F)F)ccn2)cc1C#Cc1cnc(NC(=O)N2CC(O)C2)s1]

or a pharmaceutically acceptable salt, solvate, prodrug, stereoisomer, or tautomer thereof.

In embodiments, a compound of Formula (I) or (I′) is Compound (4),

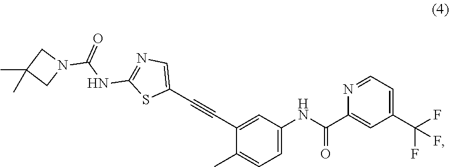 [SMILES: Cc1ccc(NC(=O)c2cc(C(F)(F)F)ccn2)cc1C#Cc1cnc(NC(=O)N2CC(C)(C)C2)s1]

or a pharmaceutically acceptable salt, solvate, prodrug, stereoisomer, or tautomer thereof.

In embodiments, a compound of Formula (I) or (I′) is Compound (5),

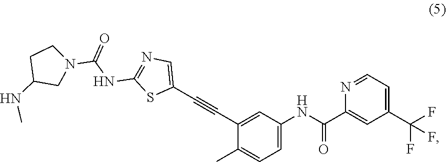 [SMILES: CNC1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, stereoisomer, or tautomer thereof.

In embodiments, a compound of Formula (I) or (I′) is Compound (5a),

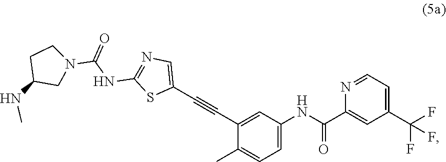 [SMILES: CN[C@H]1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, or tautomer thereof.

In embodiments, a compound of Formula (I) or (I′) is Compound (5b),

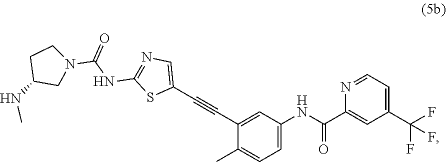 [SMILES: CN[C@@H]1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, or tautomer thereof.

A second aspect of the invention relates to a method of treating a c-Kit-mediated disease or disorder. The method comprises administering to a patient in need thereof an effective amount of a compound of Formula (I), or a pharmaceutically acceptable salt, hydrate, solvate, prodrug, stereoisomer, or tautomer thereof.

Another aspect of the invention relates to a method of preventing a c-Kit-mediated disease or disorder. The method comprises administering to a patient in need thereof an effective amount of a compound of Formula (I), or a pharmaceutically acceptable salt, hydrate, solvate, prodrug, stereoisomer, or tautomer thereof.

Another aspect of the invention relates to a method of inhibiting c-Kit. The method comprises administering to a patient in need thereof an effective amount of a compound of Formula (I), or a pharmaceutically acceptable salt, hydrate, solvate, prodrug, stereoisomer, or tautomer thereof.

Another aspect of the invention relates to a method of treating a disease or disorder associated with inhibiting c-Kit. The method comprises administering to a patient in need thereof an effective amount of a compound of Formula (I), or a pharmaceutically acceptable salt, hydrate, solvate, prodrug, stereoisomer, or tautomer thereof.

Another aspect of the invention relates to a method of preventing a disease or disorder associated with inhibiting c-Kit. The method comprises administering to a patient in need thereof an effective amount of a compound of Formula (I), or a pharmaceutically acceptable salt, hydrate, solvate, prodrug, stereoisomer, or tautomer thereof.

Another aspect of the invention relates to a method of treating cancer. The method comprises administering to a patient in need thereof an effective amount of a compound of Formula (I), or a pharmaceutically acceptable salt, hydrate, solvate, prodrug, stereoisomer, or tautomer thereof.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the present invention relates to a compound of Formula (I), or a pharmaceutically acceptable salt, hydrate, solvate, prodrug, stereoisomer, or tautomer thereof, for use in the manufacture of a medicament for treating a disease associated with inhibiting c-Kit.

Another aspect of the present invention relates to a compound of Formula (I), or a pharmaceutically acceptable salt, hydrate, solvate, prodrug, stereoisomer, or tautomer thereof, for use in the manufacture of a medicament for preventing a disease associated with inhibiting c-Kit.

Another aspect of the present invention relates to the use of a compound of Formula (I), or a pharmaceutically acceptable salt, hydrate, solvate, prodrug, stereoisomer, or tautomer thereof, in the treatment of a disease associated with inhibiting c-Kit.

Another aspect of the present invention relates to the use of a compound of Formula (I), or a pharmaceutically acceptable salt, hydrate, solvate, prodrug, stereoisomer, or tautomer thereof, in the prevention of a disease associated with inhibiting c-Kit.

The present invention further provides methods of treating or preventing a disease or disorder associated with modulation of c-Kit including, cancer, metastasis, inflammation and auto-immune pathogenesis, comprising administering to a patient suffering from at least one of said diseases or disorder a compound of Formula (I), or a pharmaceutically acceptable salt, hydrate, solvate, prodrug, stereoisomer, or tautomer thereof.

The present invention provides inhibitors of c-Kit that are therapeutic agents in the treatment of diseases such as cancer, metastasis, inflammation and auto-immune pathogenesis.

The present disclosure provides agents with novel mechanisms of action toward c-Kit enzymes in the treatment of various types of diseases including cancer and cell proliferative disorders, multiple sclerosis, asthma, mastocytosis, inflammatory disorders, allergic reactions, fibrotic disorders, auto-immune pathogenesis and metabolic disorders. Ultimately the present invention provides the medical community with a novel pharmacological strategy for the treatment of diseases and disorders associated with c-Kit.

### BRIEF DESCRIPTION OF DRAWINGS

FIG. 1 illustrates pharmacokinetic studies of Compound (1a) in rats.

FIG. 2A illustrates pharmacokinetic studies of Compound (1a) in male monkeys, and FIG. 2B illustrates pharmacokinetic studies of Compound (1a) in female monkeys.

### DETAILED DESCRIPTION OF THE INVENTION

The present invention relates to compounds and compositions that are capable of inhibiting the activity of c-Kit. In particular, compounds described herein, or a pharmaceutically acceptable salt, hydrate, solvate, prodrug, stereoisomer, or tautomer thereof, can have unexpectedly beneficial pharmacokinetic or therapeutic properties.

Accordingly, the invention also features methods of treating, preventing or ameliorating a disease or disorder in which c-Kit plays a role by administering to a patient in need thereof a therapeutically effective amount of a compound of Formula (I), or a pharmaceutically acceptable salt, hydrate, solvate, prodrug, stereoisomer, or tautomer thereof. The methods of the present invention can be used in the treatment of a variety of c-Kit dependent diseases and disorders by inhibiting the activity of c-Kit enzymes. Inhibition of c-Kit provides a novel approach to the treatment, prevention, or amelioration of diseases including, but not limited to, cancer and metastasis.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O-(C₁-C₆) alkyl, (C₁-C₆) alkyl, (C₁-C₆) alkoxy, (C₁-C₆) haloalkyl, (C₁-C₆) haloalkoxy, —O-(C₂-C₆) alkenyl, —O-(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O-(C₁-C₆) alkyl, (C₁-C₆) alkyl, —O-(C₂-C₆) alkenyl, —O-(C₂-C₆) alkynyl. (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, NH₂, NH((C₁-C₆) alkyl), N((C₁-C₆) alkyl)₂, —S(O)₂—(C₁-C₆) alkyl, —S(O)NH(C₁-C₆) alkyl, and S(O)N((C₁-C₆) alkyl)₂. The substituents can themselves be optionally substituted. Furthermore when containing two fused rings the aryl groups herein defined may have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic aromatic radical of 5 to 24 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ²-pyrrolo[2, 1-b]pyrimidine, dibenzo[b,d] thiophene, pyridin-2-one, furo[3,2-c]pyridinyl, furo[2,3-c]pyridinyl, 1H-pyrido[3,4-b][1,4] thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6, 7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore when containing two fused rings the aryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, indolinyl, indolyl, and dihydrobenzoxanyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e . . . —O(alkyl). Examples of alkoxy groups include, without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Cycloalkyl” means monocyclic saturated carbon rings containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl.

“Heterocyclyl” or “heterocycloalkyl” monocyclic rings containing carbon and heteroatoms taken from oxygen, nitrogen, or sulfur and wherein there is not delocalized T electrons (aromaticity) shared among the ring carbon or heteroatoms. The heterocycloalkyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “hydroxyalkyl” means an alkyl group as defined above, where the alkyl group is substituted with one or more —OH groups. Examples of hydroxyalkyl groups include HO—CH₂—, HO—CH₂—CH₂— and CH₃—CH(OH)—.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkyl groups include, but are not limited to, trifluoromethoxy, difluoromethoxy, pentafluoroethoxy, trichloromethoxy, etc.

The term “amine” as used herein refers to primary (R—NH₂, R≠H), secondary (R₂—NH, R₂≠H) and tertiary (R₃—N, R≠H) amines. A substituted amine is intended to mean an amine where at least one of the hydrogen atoms has been replaced by the substituent.

The term “amino” as used herein means a substituent containing at least one nitrogen atom. Specifically, NH₂, —NH(alkyl) or alkylamino, —N(alkyl)₂ or dialkylamino, amide-, carbamide-, urea, and sulfamide substituents are included in the term “amino”.

The term “alkylamino” as used herein refers to an amino or NH₂ group where one of the hydrogens has been replaced with an alkyl group, as defined herein above, i.e., —NH(alkyl). Example of alkylamino groups include, but are not limited to, methylamino (i.e., —NH(CH₃)), ethylamino, propylamino, iso-propylamino, n-butylamino, sec-butylamino, tert-butylamino, etc.

The term “dialkylamino” as used herein refers to an amino or NH₂ group where both of the hydrogens have been replaced with alkyl groups, as defined herein above, i.e., —N(alkyl)₂. The alkyl groups on the amino group can be the same or different alkyl groups. Example of alkylamino groups include, but are not limited to, dimethylamino (i.e., —N(CH₃)₂), diethylamino, dipropylamino, diiso-propylamino, di-n-butylamino, di-sec-butylamino, di-tert-butylamino, methyl(ethyl)amino, methyl(butylamino), etc.

The term “oxo” as used herein refers to an “═O” group.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

As used herein, “treating” or “treat” describes the management and care of a patient for the purpose of reversing, inhibiting, or combating a disease, condition, or disorder and includes the administration of a compound of the present disclosure (i.e., a compound of Formula (I), or a pharmaceutically acceptable salt, prodrug, metabolite, polymorph or solvate thereof, to reverse the disease, condition, or disorder, eliminate the disease, condition, or disorder, or inhibit the process of the disease, condition, or disorder.

A compound of the present disclosure (e.g., a compound of Formula (I), or a pharmaceutically acceptable salt, prodrug, metabolite, polymorph or solvate thereof, can also be used to prevent a disease, condition, or disorder or one or more symptoms of such disease, condition, or disorder. As used herein, “preventing” or “prevent” describes reducing or eliminating the onset of the symptoms or complications of the disease, condition, or disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The present invention relates to compounds or pharmaceutically acceptable salts, hydrates, solvates, prodrugs, stereoisomers, or tautomers thereof, capable of inhibiting c-Kit, which are useful for the treatment of diseases and disorders associated with modulation of a c-Kit enzyme. The invention further relates to compounds, or pharmaceutically acceptable salts, hydrates, solvates, prodrugs, stereoisomers, or tautomers thereof, which are useful for inhibiting c-Kit.

### Compounds of the Invention

A first aspect of the invention relates to compounds of Formula (I):

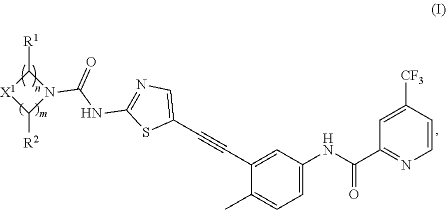

and pharmaceutically acceptable salts, hydrates, solvates, prodrugs, stereoisomers, and tautomers thereof, wherein
n and m are independently 1, 2, or 3;
X¹ is O, NR³, CR^{x}R³, or CHR³;
each R¹, R², and R³ is independently H or X²;
X² is OH, O(C₁-C₆ alkyl), NH₂, NHR⁴, or NR₄R⁵;
R⁴ and R⁵ are independently C₁-C₆ alkyl or R⁴ and R⁵, when both are present, combine to form a 4- to 6-membered heterocyclyl:
R^{x} is C₁-C₆ alkyl: and
wherein at least one of R¹, R², and R³ is X².

In embodiments, X¹ is O, NR³, or CHR³.

In embodiments, X¹ is O.

In embodiments, X¹ is NR³.

In embodiments, X¹ is CHR³.

In embodiments, X¹ is CR^{x}R³. In embodiments, R^{x} is C₁-C₆ alkyl. In embodiments, R^{x} is CH₃. In embodiments, X¹ is CR^{x}R³ and R^{x} is Me. In embodiments, X¹ is C(CH₃)R³.

In embodiments, each R¹ and R² present is H, and X¹ is NR³, or CHR³, and R³ is X².

In embodiments, each R¹ and R² present is H, and X¹ is C(CH₃)R³, and R³ is X².

In embodiments, X² is OH.

In embodiments, X² is NH₂.

In embodiments, X² is NHR⁴.

In embodiments, X² is NR⁴R⁵, and R⁴ and R⁵ combine to form a 4- to 6-membered heterocyclyl.

In embodiments, X² is NR⁴R⁵, and R⁴ and R⁵ are independently C₁-C₆ alkyl.

In embodiments, a compound of Formula (I) has a structure according to Formula (I′),

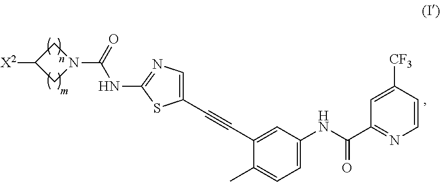

or a pharmaceutically acceptable salt, solvate, prodrug, stereoisomer, or tautomer thereof.

In embodiments, a compound of Formula (I) or (I′) has a structure according to Formula (I′-A),

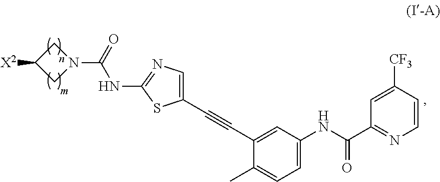

or a pharmaceutically acceptable salt, solvate, prodrug, or tautomer thereof.

In embodiments, a compound of Formula (I) or (I′) has a structure according to Formula (I′-B),

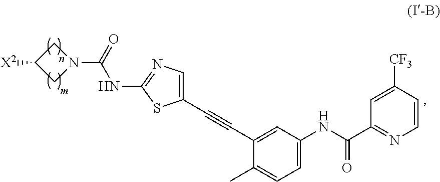

or a pharmaceutically acceptable salt, solvate, prodrug, or tautomer thereof.

In embodiments of any formula described herein, X² is OH.

In embodiments of any formula described herein, X² is NH₂, NHR⁴, or NR⁴R⁵.

In embodiments of any formula described herein, X² is OH, NH₂, NHCH₃, or N(CH₃)₂.

In embodiments of any formula described herein, X² is NH₂, NHCH₃, or N(CH₃)₂.

In embodiments of any formula described herein, X² is OH, NH₂, NHCH₃, or N(CH₃)₂.

In embodiments of any formula described herein, X² is NH₂, NHCH₃, or N(CH₃)₂.

In embodiments of any formula described herein, X² is OH.

In embodiments of any formula described herein, X² is NH₂.

In embodiment of any formula described herein, X² is NHCH₃.

In embodiments of any formula described herein, X² is N(CH₃)₂.

In embodiments of any formula described herein, n is 1. In embodiments, m is 1. In embodiments, m is 2. In embodiments, m is 3.

In embodiments of any formula described herein, n is 2. In embodiments, m is 1. In embodiments, m is 2. In embodiments, m is 3.

In embodiments of any formula described herein, n is 3. In embodiments, m is 1. In embodiments, m is 2. In embodiments, m is 3.

In embodiments of any formula described herein, m is 1. In embodiments, n is 1. In embodiments, n is 2. In embodiments, n is 3.

In embodiments of any formula described herein, m is 2. In embodiments, n is 1. In embodiments, n is 2. In embodiments, n is 3.

In embodiments of any formula described herein, m is 3. In embodiments, n is 1. In embodiments, n is 2. In embodiments, n is 3.

In embodiments of any formula described herein, n is 1, and m is 1.

In embodiments of any formula described herein, n is 1, and m is 2.

In embodiments of any formula described herein, n is 2, and m is 1.

In embodiments of any formula described herein, n is 2, and m is 2.

In embodiments of any formula described herein, m and n are each 1, and X² is OH, NH₂, NHR⁴, or NR⁴R⁵ (e.g., X² is NH₂, NHCH₃, or N(CH₃)₂).

In embodiments of any formula described herein, m+n=3, and X² is OH, NH₂, NHR⁴, or NR⁴R⁵ (e.g., X² is NH₂, NHCH₃, or N(CH₃)₂).

In embodiments of any formula described herein, m+n =4, and X² is OH, NH₂, NHR⁴, or NR⁴R⁵ (e.g., X² is NH₂, NHCH₃, or N(CH₃)₂).

In embodiments of any formula described herein. a compound of Formula (I) or (I′) is selected from the group consisting of:

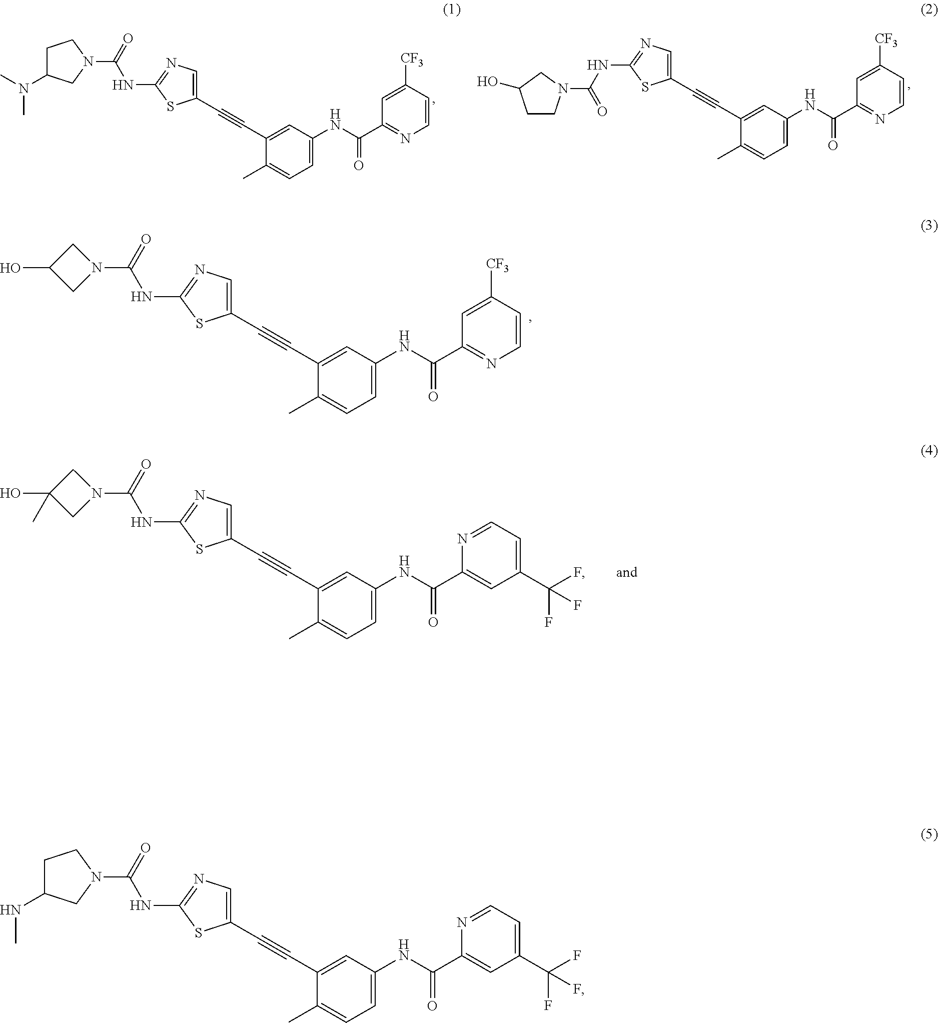 [SMILES: Cc1ccc(NC(=O)c2cc(C(F)(F)F)ccn2)cc1C#Cc1cnc(NC(=O)N2CCC(N(C)C)C2)s1 ; Cc1ccc(NC(=O)c2cc(C(F)(F)F)ccn2)cc1C#Cc1cnc(NC(=O)N2CCC(O)C2)s1 ; Cc1ccc(NC(=O)c2cc(C(F)(F)F)ccn2)cc1C#Cc1cnc(NC(=O)N2CC(O)C2)s1 ; Cc1ccc(NC(=O)c2cc(C(F)(F)F)ccn2)cc1C#Cc1cnc(NC(=O)N2CC(C)(O)C2)s1 ; "and" ; CNC1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, stereoisomer, or tautomer thereof.

In embodiments, a compound of Formula (I) or (I′) is Compound (1).

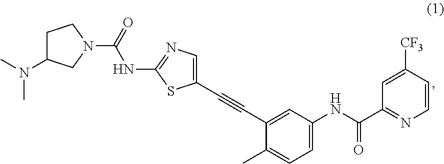 [SMILES: Cc1ccc(NC(=O)c2cc(C(F)(F)F)ccn2)cc1C#Cc1cnc(NC(=O)N2CCC(N(C)C)C2)s1]

or a pharmaceutically acceptable salt, solvate, prodrug, stereoisomer, or tautomer thereof.

In embodiments, a compound of Formula (I) or (I′) is Compound (1a),

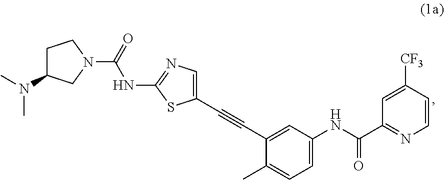 [SMILES: Cc1ccc(NC(=O)c2cc(C(F)(F)F)ccn2)cc1C#Cc1cnc(NC(=O)N2CC[C@H](N(C)C)C2)s1]

or a pharmaceutically acceptable salt, solvate, prodrug, or tautomer thereof.

In embodiments, a compound of Formula (I) or (I′) is Compound (1b),

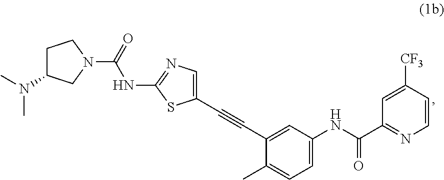 [SMILES: Cc1ccc(NC(=O)c2cc(C(F)(F)F)ccn2)cc1C#Cc1cnc(NC(=O)N2CC[C@@H](N(C)C)C2)s1]

or a pharmaceutically acceptable salt, solvate, prodrug, or tautomer thereof.

In embodiments, a compound of Formula (I) or (I′) is Compound (2),

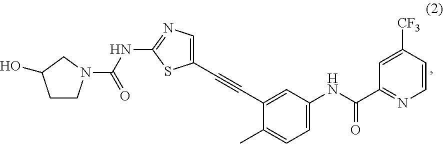 [SMILES: Cc1ccc(NC(=O)c2cc(C(F)(F)F)ccn2)cc1C#Cc1cnc(NC(=O)N2CCC(O)C2)s1]

or a pharmaceutically acceptable salt, solvate, prodrug, stereoisomer, or tautomer thereof.

In embodiments, a compound of Formula (I) or (I′) is Compound (2a),

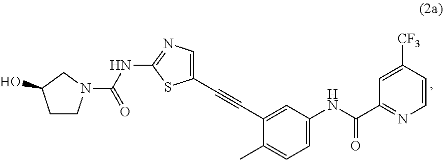 [SMILES: Cc1ccc(NC(=O)c2cc(C(F)(F)F)ccn2)cc1C#Cc1cnc(NC(=O)N2CC[C@@H](O)C2)s1]

or a pharmaceutically acceptable salt, solvate, prodrug, or tautomer thereof.

In embodiments, a compound of Formula (I) or (I′) is Compound (2b),

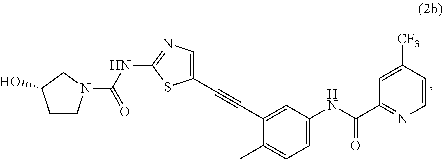 [SMILES: Cc1ccc(NC(=O)c2cc(C(F)(F)F)ccn2)cc1C#Cc1cnc(NC(=O)N2CC[C@H](O)C2)s1]

or a pharmaceutically acceptable salt, solvate, prodrug, or tautomer thereof.

In embodiments, a compound of Formula (I) or (I′) is Compound (3),

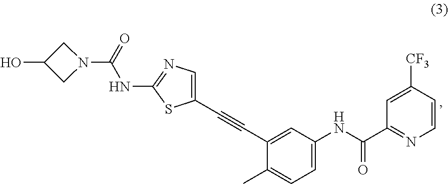 [SMILES: Cc1ccc(NC(=O)c2cc(C(F)(F)F)ccn2)cc1C#Cc1cnc(NC(=O)N2CC(O)C2)s1]

or a pharmaceutically acceptable salt, solvate, prodrug, stereoisomer, or tautomer thereof.

In embodiments, a compound of Formula (I) or (I′) is Compound (4),

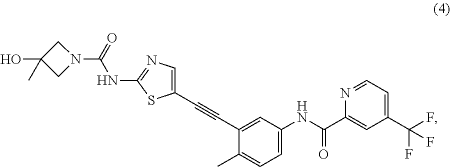 [SMILES: Cc1ccc(NC(=O)c2cc(C(F)(F)F)ccn2)cc1C#Cc1cnc(NC(=O)N2CC(C)(O)C2)s1]

or a pharmaceutically acceptable salt, solvate, prodrug, stereoisomer, or tautomer thereof.

In embodiments, a compound of Formula (I) or (I′) is Compound (5),

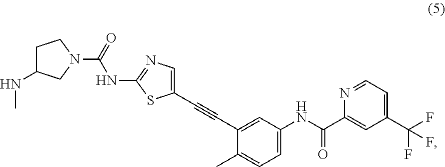 [SMILES: CNC1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, stereoisomer, or tautomer thereof.

In embodiments, a compound of Formula (I) or (I′) is Compound (5a),

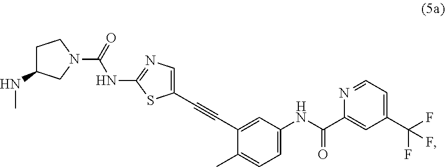 [SMILES: CN[C@H]1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, or tautomer thereof.

In embodiments, a compound of Formula (I) or (I′) is Compound (5b),

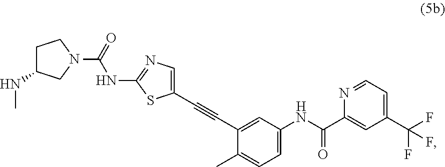 [SMILES: CN[C@@H]1CCN(C(=O)Nc2ncc(C#Cc3cc(NC(=O)c4cc(C(F)(F)F)ccn4)ccc3C)s2)C1]

or a pharmaceutically acceptable salt, solvate, prodrug, or tautomer thereof.

In another embodiment of the invention, the compounds of Formula (I) are enantiomers. In some embodiments the compounds are the (S)-enantiomer. In other embodiments the compounds are the (R)-enantiomer. In yet other embodiments, the compounds of Formula (I) may be (+) or (−) enantiomers.

It should be understood that all isomeric forms are included within the present invention, including mixtures thereof, unless otherwise specified. If the compound contains a double bond, the substituent may be in the E or Z configuration. If the compound contains a disubstituted cycloalkyl, the cycloalkyl substituent may have a cis-or trans configuration. All tautomeric forms are also intended to be included.

Compounds of the invention, and pharmaceutically acceptable salts, hydrates, solvates, stereoisomers and prodrugs thereof may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention unless otherwise specified. In addition, the present invention embraces all geometric and positional isomers. For example, if a compound of the invention incorporates a double bond or a fused ring, both the cis-and trans-forms, as well as mixtures, are embraced within the scope of the invention. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry unless otherwise specified.

In embodiments, the invention features a particular diastereomeric form of the compound. Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of a chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis-and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula (I) may form salts (e.g., pharmaceutically acceptable salts) which are also within the scope of this invention. Reference to a compound of the Formula herein is understood to include reference to salts thereof, unless otherwise indicated.

The present invention relates to compounds which are modulators of c-Kit. In one embodiment, the compounds of the present invention are inhibitors of c-Kit.

The invention is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Method of Synthesizing the Compounds

The compounds of the present invention may be made by a variety of methods, including standard methods known in the art. For example, compounds described herein may be made from commercially available starting materials or synthesized using known organic, inorganic, and/or enzymatic processes.

Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula (I).

Those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

### Methods of Using the Disclosed Compounds

Compounds described herein can be potent inhibitors of c-Kit.

Accordingly, another aspect of the invention relates to a method of treating a disease or disorder associated with modulation of c-Kit. The method comprises administering to a patient in need of a treatment for a disease or disorder associated with modulation of c-Kit an effective amount the compositions and compounds of Formula (I).

Another aspect of the invention relates to a method of preventing a disease or disorder associated with modulation of c-Kit. The method comprises administering to a patient in need of a treatment for a disease or disorder associated with modulation of c-Kit an effective amount the compositions and compounds of Formula (I).

Another aspect of the invention relates to a method of treating a c-Kit-mediated disease or disorder. The method comprises administering to a patient in need of a treatment of a disease or disorder associated with modulation of c-Kit an effective amount the compositions and compounds of Formula (I).

Another aspect of the invention relates to a method of preventing a c-Kit-mediated disease or disorder. The method comprises administering to a patient in need of a treatment for diseases or disorders associated with modulation of c-Kit an effective amount the compositions and compounds of Formula (I).

In another aspect, the present invention is directed to a method of inhibiting c-Kit. The method involves administering to a patient in need thereof an effective amount of a compound of Formula (I).

Another aspect of the present invention relates to a method of treating a disease or disorder in a patient associated with the inhibition of c-Kit, the method comprising administering to a patient in need thereof an effective amount of a compound of Formula (I). In one embodiment, the disease or disorder is selected from the group consisting of cancer and cell proliferative disorders, multiple sclerosis, asthma, mastocytosis, inflammatory disorders, allergic reactions, fibrotic disorders, and metabolic disorders.

Another aspect of the present invention relates to a method of preventing a disease or disorder in a patient associated with the inhibition of c-Kit, the method comprising administering to a patient in need thereof an effective amount of a compound of Formula (I).

The present invention also relates to the use of an inhibitor of c-Kit for the preparation of a medicament used in the treatment, prevention, inhibition or elimination of a disease or disorder mediated by c-Kit, wherein the medicament comprises a compound of Formula (I).

In another aspect, the present invention relates to a method for the manufacture of a medicament for treating, preventing, inhibiting, or eliminating a disease or disorder mediated by c-Kit, wherein the medicament comprises a compound of Formula (I).

Another aspect of the present invention relates to a compound of Formula (I) for use in the manufacture of a medicament for treating a disease or disorder associated with inhibiting c-Kit.

In another aspect, the present invention relates to the use of a compound of Formula (I) in the treatment of a disease or disorder associated with inhibiting c-Kit.

In another aspect, the present invention relates to the use of a compound of Formula (I) in the prevention of a disease or disorder associated with inhibiting c-Kit.

In some embodiments of the methods above, the disease or disorder is selected from the group consisting of cancer, metastasis, inflammation and auto-immune pathogenesis.

In some embodiments of the methods above, the disease or disorder is selected from the group consisting of cell proliferative disorder, a fibrotic disorder, and a metabolic disorder.

In an embodiment of the methods above, the disease or disorder is multiple sclerosis.

In an embodiment of the methods above, the disease or disorder is asthma. In another embodiment of the methods above, the disease or disorder is mastocytosis.

In an embodiment of the methods above, the disease or disorder is an allergic reaction.

In an embodiment of the methods above, the disease or disorder is inflammatory arthritis.

Another aspect of the invention relates to a method of treating cancer. The method comprises administering to a patient in need thereof an effective amount of a compound of Formula (I).

In some embodiments, the cancer is selected from liposarcoma, neuroblastoma, glioblastoma, bladder cancer, adrenocortical cancer, multiple myeloma, colorectal cancer, non-small cell lung cancer, oropharyngeal cancer, penis cancer, anal cancer, thyroid cancer, vaginal cancer, gastric cancer, rectal cancer, thyroid cancer, Hodgkin lymphoma and diffuse large B-cell lymphoma.

In some embodiments, the cancer is selected from leukemia, mast cell tumor, small cell lung cancer, testicular cancer, cancer of the gastrointestinal tract, cancer of the central nervous system, cancer of the female genital tract, sarcoma of neuroectodermal origin, and Schwann cell neoplasia associated with neurofibromatosis.

In some embodiments, the cancer is selected from small cell lung carcinoma, acute myeloid leukemia (AML), thymic carcinoma, desmoid tumor, neuroblastoma, malignant melanomas, colorectal cancer, systemic mastocytosis (SM), and gastrointestinal stromal tumors (GISTs).

Another aspect of the invention relates to a method of inducing cell cycle arrest, apoptosis in tumor cells, and/or enhanced tumor-specific T cell immunity. The method comprises contacting the cells with an effective amount of a compound of Formula (I).

In one embodiment, the present invention relates to the use of an inhibitor of c-Kit for the preparation of a medicament used in treatment, prevention, inhibition or elimination of a disease or disorder associated with associated with cancer and metastasis.

In some embodiments, administration of a compound of Formula (I) or a pharmaceutical composition comprising a compound of the present invention and a pharmaceutically acceptable carrier induces a change in the cell cycle or cell viability.

Another aspect of the invention relates to a method of treating inflammation. The method comprises administering to a patient in need thereof an effective amount of a compound of Formula (I).

Another aspect of the invention relates to a method of treating auto-immune pathogenesis. The method comprises administering to a patient in need thereof an effective amount of a compound of Formula (I).

Another aspect of the invention is directed to pharmaceutical compositions comprising a compound of Formula (I) and a pharmaceutically acceptable carrier. The pharmaceutical acceptable carrier may further include an excipient, diluent, or surfactant.

In one embodiment, are provided methods of treating a disease or disorder associated with modulation of c-Kit including, cancer, metastasis, inflammation and auto-immune pathogenesis, comprising administering to a patient suffering from at least one of said diseases or disorder a compound of Formula (I).

In one embodiment, are provided methods of treating a disease or disorder associated with modulation of c-Kit including, cancer and metastasis, comprising administering to a patient suffering from at least one of said diseases or disorder a compound of Formula (I).

One therapeutic use of the compounds or compositions of the present invention which inhibit c-Kit is to provide treatment to patients or subjects suffering from cancer, metastasis, inflammation and auto-immune pathogenesis.

Another therapeutic use of the compounds or compositions of the present invention which inhibit c-Kit is to provide treatment to patients or subjects suffering from cancer and metastasis.

The disclosed compounds of the invention can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine: b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol: for tablets also: c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired: d) a disintegrant, e.g., starches, agar, methyl cellulose, bentonite, xanthan gum, algic acid or its sodium salt, or effervescent mixtures: e) absorbent, colorant, flavorant and sweetener: f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier: and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions: using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention is directed to pharmaceutical compositions comprising a compound of Formula (I) and a pharmaceutically acceptable carrier. The pharmaceutical acceptable carrier may further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient: the severity of the condition to be treated: the route of administration; the renal or hepatic function of the patient: and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Analytical Methods, Materials, and Instrumentation

Unless otherwise noted, reagents and solvents were used as received from commercial suppliers. Proton nuclear magnetic resonance (NMR) spectra were obtained on Bruker spectrometers at 400 MHz. Spectra are given in ppm (δ) and coupling constants, J, are reported in Hertz. Tetramethylsilane (TMS) was used as an internal standard. Mass spectra were collected using a Waters ZQ Single Quad Mass Spectrometer (ion trap electrospray ionization (ESI)). Purity and low resolution mass spectral data were measured using Waters Acquity i-class ultra-performance liquid chromatography (UPLC) system with Acquity Photo Diode Array Detector, Acquity Evaporative Light Scattering Detector (ELSD) and Waters ZQ Mass Spectrometer. Data was acquired using Waters MassLynx 4.1 software and purity characterized by UV wavelength 220 nm, evaporative light scattering detection (ELSD) and electrospray positive ion (ESI). (Column: Acquity UPLC BEH C18 1.7 μm 2.1×50 mm: Flow rate 0.6 mL/min: Solvent A (95/5/0.1%: 10 mM Ammonium Formate/Acetonitrile/Formic Acid), Solvent B (95/5/0.09%: Acetonitrile/Water/Formic Acid); gradient: 5-100% B from 0 to 2 mins, hold 100% B to 2.2 mins and 5% B at 2.21 mins. Abbreviations used in the following examples and elsewhere herein are:

| | |
|---|---|
| br | broad |
| CDI | 1,1′-carbonyldiimidazole |
| DCM | dichloromethane |
| DIPEA | N,N-diisopropylethylamine |
| DMAP | 4-(dimethylamino)pyridine |
| DMF | N,N-dimethylformamide |
| DMSO | dimethyl sulfoxide |
| EI | electron ionization |
| ESI | electrospray ionization |
| Et₃N | triethylamine |
| EtOAc | ethyl acetate |
| EtOH | ethanol |
| GCMS | gas chromatography-mass spectrometry |
| h | hour(s) |
| HATU | 2-(7-Aza-1H-benzotriazole-1-yl)-1,1,3,3- |
| | tetramethyluronium hexafluorophosphate |
| HPLC | high-performance liquid chromatography |
| LCMS | liquid chromatography-mass spectrometry |
| m | multiplet |
| Me | methyl |
| MeOH | methanol |
| MHz | megahertz |
| min | minutes |
| MS | molecular sieves |
| NMR | nuclear magnetic resonance |
| ppm | parts per million |
| PSI | Pounds per square inch |
| s | singlet |
| TBAF | tetra-n-butylammonium fluoride |
| TFA | trifluoroacetic acid |
| THF | tetrahydrofuran |

### Example 1. Synthesis of Exemplary Compounds

Preparation of Compound 1a: N-[3-[2-[2-[[(3S)-3-(dimethylamino)pyrrolidine-1 -carbonyl]amino]thiazol-5-yl]ethynyl]-4-methyl-phenyl]-4-(trifluoromethyl)pyridine-2-carboxamide

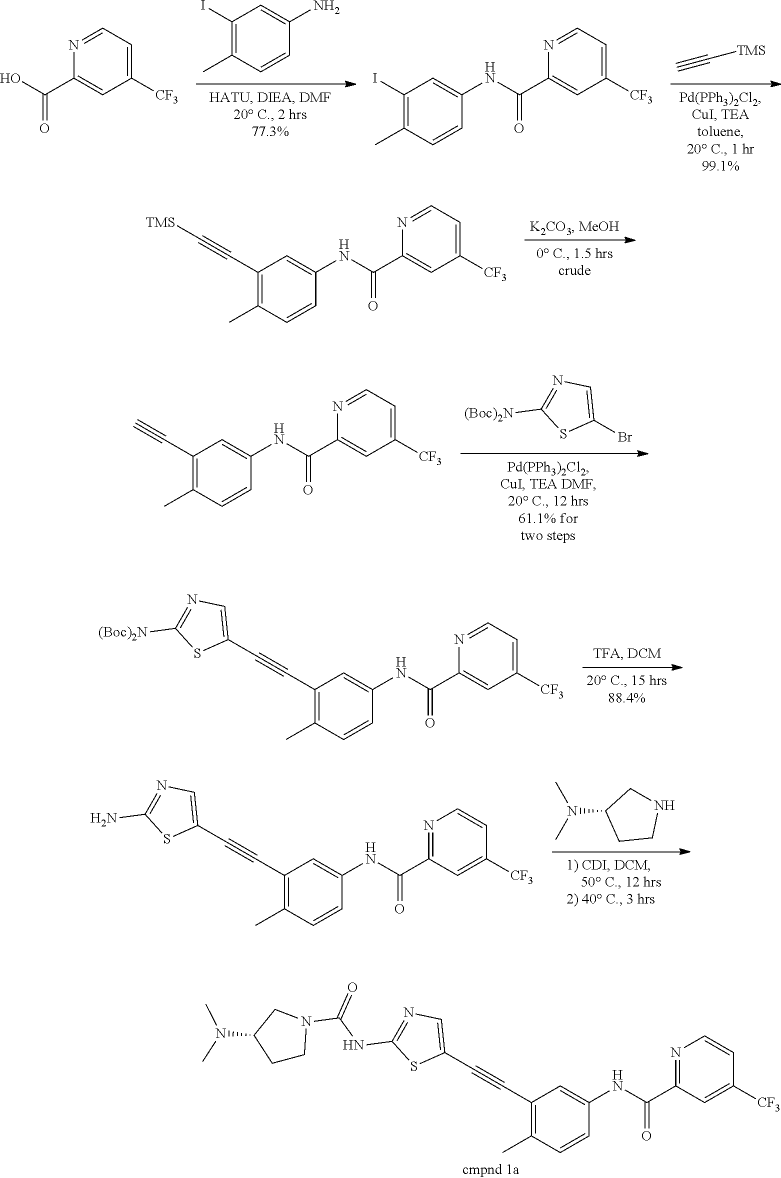

### Step 1: Synthesis of N-(3-iodo-4-methyl-phenyl)-4-(trifluoromethyl)pyridine-2-carboxamide

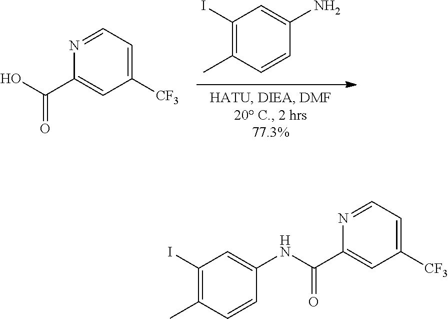

A mixture of 4-(trifluoromethyl)pyridine-2-carboxylic acid (5 g, 26.2 mmol, 1.0 eq), HATU (15 g, 39.5 mmol, 1.5 eq) and DIEA (25 mL, 0.144 mmol, 5.5 eq) in DMF (100 mL) was stirred at 20° C. for 30 minutes. Then 3-iodo-4-methyl-aniline (6.1 g, 26.2 mmol, 1.0 eq) and the mixture was stirred at 20° C. for 1.5 hours. The reaction mixture was quenched by addition of water (150 mL) and extracted with EtOAc (80 mL*3). The combined organic phase was washed with brine (100 mL*5), dried with anhydrous Na₂SO₄, filtered and concentrated in vacuum. The residue was purified by column chromatography (SiO₂, petroleum ether/EtOAc=100/1 to 30/1, 254 nm) to afford N-(3-iodo-4-methyl-phenyl)-4 -(trifluoromethyl)pyridine-2-carboxamide (8.3 g, 77.3% yield, 99% purity) as a white solid.

### Step 2: Synthesis of N-[4-methyl-3-(2-trimethylsilylethynyl)phenyl]-4-(trifluoromethyl)pyridine-2-carboxamide

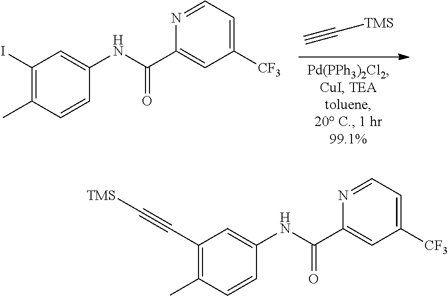

N-(3-iodo-4-methyl-phenyl)-4-(trifluoromethyl)pyridine-2-carboxamide (8.3 g, 20.4 mmol, 1 eq), ethynyl(trimethyl)silane (14 mL, 0.101 mol, 4.95 eq), Cul (800 mg, 4.20 mmol, 0.21 eq), Pd(PPh₃)₂Cl₂ (1.5 g, 2.14 mmol, 0.11 eq) and TEA (10 mL, 71.9 mmol, 3.52 eq) in toluene (100 mL) was degassed and then heated to 20° C. for 1 hour under nitrogen. The reaction was filtered and washed with EtOAc (20 mL*3). The combined filtrate was concentrated under reduced pressure and the residue was purified by column chromatography (SiO₂, petroleum ether/EtOAc=100/1 to 60/1, 254 nm) to afford N-[4-methyl-3-(2 -trimethylsilylethynyl)phenyl]-4-(trifluoromethyl)pyridine-2-carboxamide (7.7 g, 99.1% yield, 99% purity) as a yellow solid.

### Step 3: Synthesis of N-(3-ethynyl-4-methyl-phenyl)-4-(trifluoromethyl)pyridine-2-carboxamide

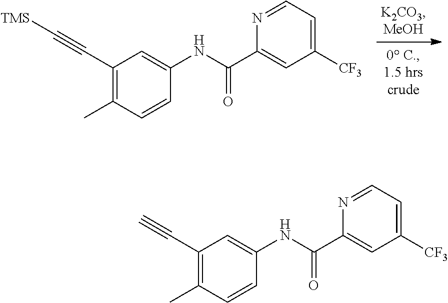 [SMILES: Cc1ccc(NC(=O)c2cc(C(F)(F)F)ccn2)cc1C#C[Si](C)(C)C ; C#Cc1cc(NC(=O)c2cc(C(F)(F)F)ccn2)ccc1C]

To a solution of N-[4-methyl-3-(2-trimethylsilylethynyl)phenyl]-4 -(trifluoromethyl)pyridine-2-carboxamide (14.8 g, 39.3 mmol, 1.0 eq) in MeOH (250 mL) was added K₂CO₃ (5.5 g, 39.8 mmol, 1.0 eq). The mixture was stirred at 0° C. for 1.5 hours. The reaction mixture was concentrated under reduced pressure and water (300 mL) was added. The mixture was extracted with EtOac (200 mL*3) and the combined organic phase was washed with brine (500 mL), dried with anhydrous Na₂SO₄, filtered and concentrated in vacuum to give N-(3-ethynyl-4-methyl-phenyl)-4-(trifluoromethyl)pyridine-2-carboxamide (11 g, crude) as a brown solid.

### Step 4: Synthesis of tert-butyl N-tert-butoxycarbonyl-N-[5-[2-[2-methyl-5-[[4 -(trifluoromethyl)pyridine-2-carbonyl]amino]phenyl]ethynyl]thiazol-2-yl]carbamate

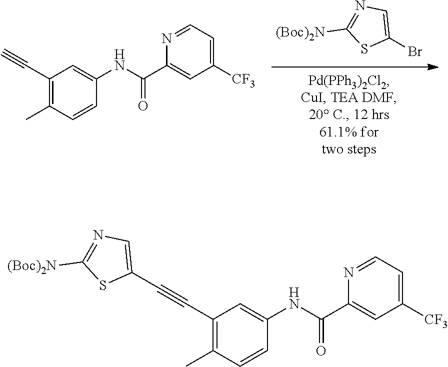

A mixture of tert-butyl N-(5-bromothiazol-2-yl)-N-tert-butoxycarbonyl-carbamate (11 g, 29 mmol, 1.0 eq), N-(3-ethynyl-4-methyl-phenyl)-4-(trifluoromethyl)pyridine-2-carboxamide (11 g, 36.2 mmol, 1.25 eq), Pd(PPh₃)₂Cl₂ (2.04 g, 2.90 mmol, 0.1 eq), CuI (1.10 g, 5.80 mmol, 0.2 eq) and TEA (17 mL, 0.122 mol, 4.21 eq) in DMF (200 mL) was degassed and purged with nitrogen for 3 times, and then the mixture was stirred at 20° C. for 12 hours under nitrogen atmosphere. The reaction mixture was concentrated in vacuum and water (500 mL) was added. The mixture was extracted with EtOAc (300 mL*3) and the combined organic phase was washed with brine (500 mL*2), dried with anhydrous Na₂SO₄, filtered and concentrated under reduced pressure. The residue was purified by silica gel chromatography (silica, petroleum ether/EtOAc=1/0 to 10/1, 254nm) to give tert-butyl N-tert-butoxycarbonyl-N-[5-[2-[2-methyl-5-[[4-(trifluoromethyl)pyridine-2-carbonyl]amino]phenyl]ethynyl]thiazol-2-yl]carbamate (11 g, 61.1% yield, 97% purity) as a yellow solid.

### Step 5: Synthesis of N-[3-[2-(2-aminothiazol-5-yl)ethynyl]-4-methyl-phenyl]-4 -(trifluoromethyl)pyridine-2-carboxamide

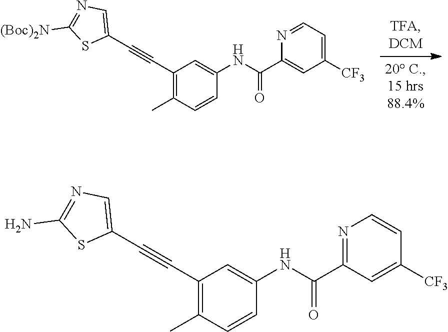

To a solution of tert-butyl N-tert-butoxycarbonyl-N-[5-[2-[2-methyl-5-[[4-(trifluoromethyl)pyridine-2-carbonyl]amino]phenyl]ethynyl]thiazol-2-yl]carbamate (11 g, 18.3 mmol, 1.0 eq) in DCM (50 mL) was added TFA (30 mL, 0.405 mol, 22.0 eq) at 0° C. The mixture was stirred at 20° C. for 15 hours. The reaction mixture was quenched by addition of saturated NaHCO₃ aqueous solution (300 mL) at 0° C. and extracted with EtOAc (200 mL* 2). The combined organic phase was washed with brine (500 mL*2), dried with anhydrous Na₂SO₄, filtered and concentrated in vacuum to give N-[3-[2-(2-aminothiazol-5-yl)ethynyl]-4-methyl-phenyl]-4-(trifluoromethyl)pyridine-2-carboxamide (6.5 g, 88.4%) as a yellow solid.

### Step 6: Synthesis of N-[3-[2-[2-[[(3S)-3-(dimethylamino)pyrrolidine-1-carbonyl]amino]thiazol-5-yl]ethynyl]-4-methyl-phenyl]-4-(trifluoromethyl)pyridine-2-carboxamide

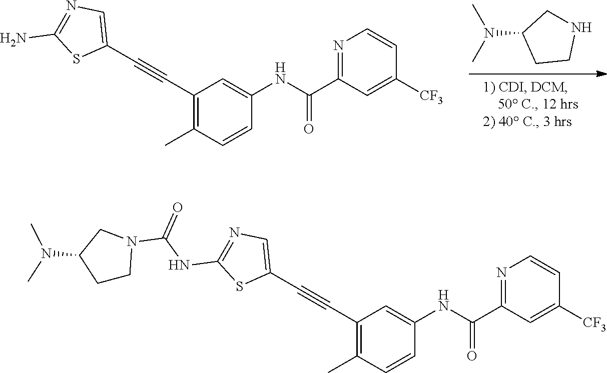

A mixture of N-[3-[2-(2-aminothiazol-5-yl)ethynyl]-4-methyl-phenyl]-4 -(trifluoromethyl)pyridine-2-carboxamide (50 mg, 0.12 mmol, 1.0 eq) and CDI (40 mg, 0.25 mmol, 2.0 eq) in DCM (5.0 mL) was stirred at 50° C. for 12 hours. Then to the mixture was added (3S)-N,N-dimethylpyrrolidin-3-amine (60 mg, 0.53 mmol, 4.0 eq) and the mixture was stirred at 40° C. for 3 hours. LCMS showed desired compound. The reaction mixture was concentrated under reduced pressure to give a residue. The residue was purified by prep-HPLC (Column: Xtimate C18 150*25 mm*5 um:Mbile phase: [water(0.05% HCl)-ACN]; B %: 22%-52%, 8.5 min, Column Temp.30° C.). Compound N-[3-[2-[2-[[(3S)-3 -(dimethylamino)pyrrolidine-1-carbonyl]amino]thiazol-5-yl]ethynyl]-4 -methyl-phenyl]-4-(trifluoromethyl)pyridine-2-carboxamide; hydrochloride (45.6 mg, 0.08 mmol, 62.7% yield, 99% purity) was obtained as a white solid.

The syntheses of compound 1b, 2a, 2b, 3, 4 and 5a (Table 1) followed the same chemical sequence by substituting appropriate secondary amines for dimethylaminopyrrolidine in the final step. Subsequent Boc deprotection with TFA was conducted for the synthesis of compound 5a.

TABLE 1

| | | | ESI-MS |
|---|---|---|---|
| Cmpnd | | | m/z |
| number | Structure | ¹H NMR (400 MHz): δ ppm | [M + H]⁺ |
| 1a | 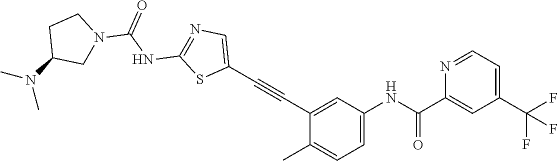 [SMILES: Cc1ccc(NC(=O)c2cc(C(F)(F)F)ccn2)cc1C#Cc1cnc(NC(=O)N2CC[C@H](N(C)C)C2)s1] | (CD3OD) δ 8.98 (d, J = 5.2 Hz, 1H), 8.44 (s, 1H), 8.04 (d, J = 2.4 Hz, 1H), 7.95-7.91 (m, 1H), 7.71-7.67 (m, 2H), 7.32 (d, J = 8.4 Hz, 1H), 4.10- 3.99 (m, 2H), 3.90-3.82 (m, 1H), 3.78-3.72 (m, 1H), 3.67- 3.58 (m, 1H), 2.98 (s, 6H), 2.55 (br d, J = 3.8 Hz, 1H), 2.47 (s, 3H), 2.38-2.27 (m, 1H) | 543.1 |
| 1b | 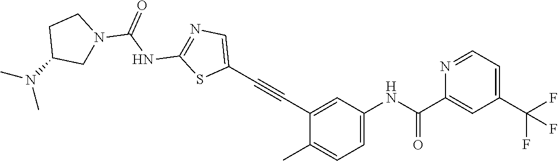 [SMILES: Cc1ccc(NC(=O)c2cc(C(F)(F)F)ccn2)cc1C#Cc1cnc(NC(=O)N2CC[C@@H](N(C)C)C2)s1] | (CD3OD) δ 8.97 (d, J = 5.1 Hz, 1H), 8.44 (s, 1H), 8.01 (d, J = 2.4 Hz, 1H), 7.93 (d, J = 4.1 Hz, 1H), 7.68 (dd, J = 2.3, 8.3 Hz, 1H), 7.62 (s, 1H), 7.31 (d, J = 8.4 Hz, 1H), 4.09- 3.97 (m, 2H), 3.87-3.79 (m, 1H), 3.71 (qd, J = 5.0, 9.6 Hz, 1H), 3.64-3.56 (m, 1H), 2.98 (s, 6H), 2.55 (br d, J = 5.3 Hz, 1H), 2.46 (s, 3H), 2.34-2.25 | 543.1 |
| | | (m, 1H) | |
| 2a | 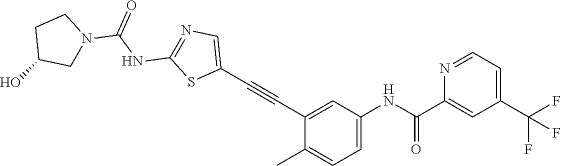 [SMILES: Cc1ccc(NC(=O)c2cc(C(F)(F)F)ccn2)cc1C#Cc1cnc(NC(=O)N2CC[C@@H](O)C2)s1] | (CD3OD) δ 8.97 (d, J = 5.0 Hz, 1H), 8.43 (s, 1H), 8.01 (d, J = 2.4 Hz, 1H), 7.92 (dd, J = 1.2, 5.0 Hz, 1H), 7.69 (dd, J = 2.4, 8.4 Hz, 1H), 7.67 (s, 1H), 7.30 (d, J = 8.4 Hz, 1H), 4.49 (br s, 1H), 3.65 (dd, J = 5.0, 9.2 Hz, 2H), 3.59 (br s, 1H), 3.54-3.47 (m, 1H), 2.46 (s, 3H), 2.17-1.92 (m, 2H) | 516.0 |
| 2b | 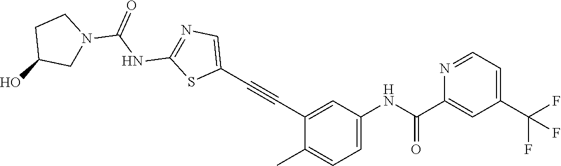 [SMILES: Cc1ccc(NC(=O)c2cc(C(F)(F)F)ccn2)cc1C#Cc1cnc(NC(=O)N2CC[C@H](O)C2)s1] | (CD3OD) δ 8.97 (d, J = 4.8 Hz, 1H), 8.43 (s, 1H), 8.02 (d, J = 2.0 Hz, 1H), 7.92 (d, J = 4.4 Hz, 1H), 7.73-7.66 (m, 2H), 7.30 (d, J = 8.4 Hz, 1H), 4.49 (br s, 1H), 3.65 (dd, J = 4.8, 9.2 Hz, 2H), 3.55-3.47 (m, 1H), 2.46 (s, 3H) | 516.0 |
| 3 | 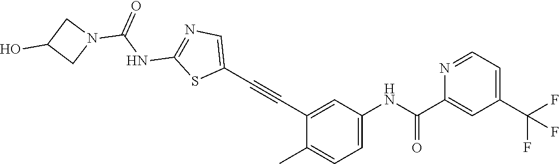 [SMILES: Cc1ccc(NC(=O)c2cc(C(F)(F)F)ccn2)cc1C#Cc1cnc(NC(=O)N2CC(O)C2)s1] | (CD3OD) δ 8.96 (d, J = 5.2 Hz, 1H), 8.42 (s, 1H), 8.00 (d, J = 2.4 Hz, 1H), 7.91 (dd, J = 1.2, 5.2 Hz, 1H), 7.69-7.63 (m, 2H), 7.29 (d, J = 8.4 Hz, 1H), 4.63 (tt, J = 4.4, 6.8 Hz, 1H), 4.41-4.30 (m, 2H), 3.94 (dd, J = 4.4, 9.2 Hz, 2H), 2.45 (s, 3H) | 502.0 |
| 4 | 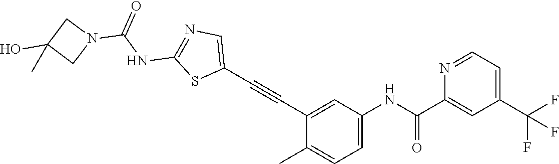 [SMILES: Cc1ccc(NC(=O)c2cc(C(F)(F)F)ccn2)cc1C#Cc1cnc(NC(=O)N2CC(C)(O)C2)s1] | (CD3OD) δ 8.96 (d, J = 5.2 Hz, 1H), 8.42 (s, 1H), 8.00 (d, J = 2.4 Hz, 1H), 7.93-7.90 (m, 1H), 7.70-7.63 (m, 2H), 7.29 (d, J = 8.4 Hz, 1H), 4.05- 3.98 (m, 4H), 2.45 (s, 3H), 1.51 (s, 3H) | 516.0 |
| 5a | 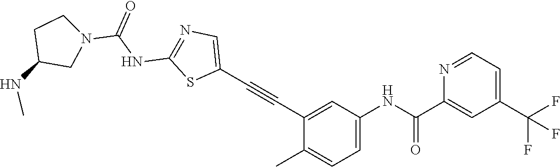 [SMILES: CN[C@H]1CCN(C(=O)Nc2ncc(C#Cc3cc(NC(=O)c4cc(C(F)(F)F)ccn4)ccc3C)s2)C1] | (CD3OD) δ 8.99 (d, J = 5.2 Hz, 1H), 8.46 (s, 1H), 7.99 (d, J = 2.0 Hz, 1H), 7.94 (d, J = 4.8 Hz, 1H), 7.71 (dd, J = 2.4, 8.3 Hz, 1H), 7.58 (s, 1H), 7.32 (d, J = 8.4 Hz, 1H), 3.75- 3.63 (m, 2H), 3.60-3.52 (m, 1H), 3.42-3.36 (m, 2H), 2.48 (s, 3H), 2.43 (s, 3H), 2.23 (br d, J = 5.2 Hz, 1H), 1.91 (br s, 1H) | 529.0 |

### Example 2. Biochemical Assays

### c-Kit Assay

### Generation of Ba/F3 KIT Mutant Engineered Cell Lines

KIT cDNAs were synthesized by GenScript and cloned into the pLVX-IRES-Puro vector (Clontech). Viral particles were produced by transfecting pLVX-IRES-puro vectors containing KIT mutant genes into HEK293 cells (Invitrogen) using the Trans-Lentiviral ORF Packaging Kit (Thermo Scientific). 48 hours post-transfection, virus-containing supernatants were harvested and incubated for another 48-72 hours with parental Ba/F3 cells (DSMZ) in the presence of 10 ng/ml IL-3 (R&D Systems). Transduced Ba/F3 cells were then selected by IL-3 withdrawal and puromycin (0.5-1 μg/mL, Invitrogen).

### Viability Assays

Cell lines (e.g., EX11DEL, EX11KEL/D816H, EX11DEL/T670I, and EX11DEL/V654A) were plated into 384 well plates using RPMI 1640 supplemented with 10% FBS at densities that produced linear growth and incubated at 37° C. in 5%(v/v) CO₂. Cells were treated with eight concentrations of compound over a 4-fold dilution (10 uM to 0.61 nM) and viability was assessed using Cell Titer-Glo assay (Promega) after 72 hours. Data were plotted as percent viability relative to vehicle-treated cells. Dose-responses curves were generated and used to calculate IC₅₀ values.

Table 2: c-Kit activity of compounds of the invention in the c-Kit assay.++++ indicates an IC₅₀ of less than about 10 nM, +++ indicates an IC₅₀ between about 10 nM and about 50 nM, ++ indicates an IC₅₀ between about 50 nM and about 100 nM, and + indicates an IC₅₀ greater than about 100 nM and less than about 10 μM.

TABLE 2
cKit Assay.

| | | BAF3 KIT | BAF3 KIT | BAF3 KIT |
|---|---|---|---|---|
| Compound | BAF3 KIT | EX11 DEL/ | EX11 DEL/ | EX11 DEL/ |
| Number | EX11 DEL | D816H | T670I | V654A |
| 1a | ++++ | +++ | +++ | +++ |
| 1b | ++++ | +++ | +++ | +++ |
| 2a | ++++ | ++++ | ++++ | ++++ |
| 2b | ++++ | ++++ | ++++ | ++++ |
| 3 | ++++ | +++ | +++ | +++ |
| 4 | ++++ | +++ | +++ | +++ |
| 5a | ++++ | +++ | ++ | +++ |

### Example 3. In Vivo Pharmacokinetic Studies of Compound (1a)

Following in vivo studies, administration of compounds of the invention can afford unexpectedly improved pharmacokinetic effects, including as compared to other aminothiazole c-Kit inhibitors.

Following in vivo studies, administration of compounds of the invention can afford unexpectedly improved pharmacokinetic effects, including as compared to other aminothiazole c-Kit inhibitors.

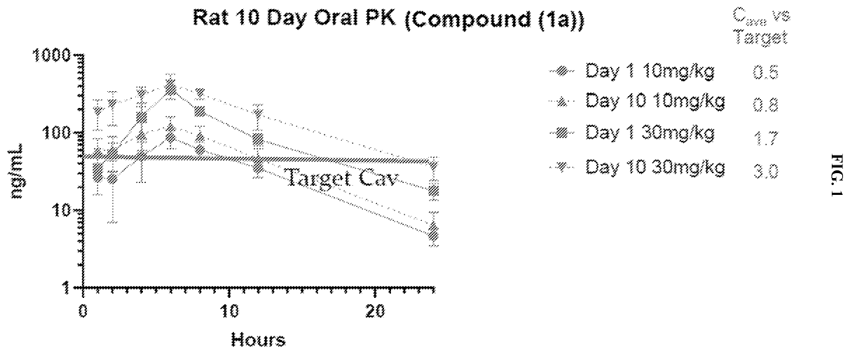

For example, the pharmacokinetics of Compound (1a) has been studied. In single dose rat pharmacokinetic studies, high clearance (Cl) and volume of distribution (V_{d}) were observed in this species. Further, two different doses (10 mg/kg and 30 mg/kg) were orally administered to rats, the average observed plasma concentration (Cₐᵥ) exceeded target exposure required for achieving desired antitumor efficacy when using the latter dose. After repeat dosing for 10 days, increased AUC (area under the curve) were obtained with Compound (1a) at either dose (see FIG. 1).

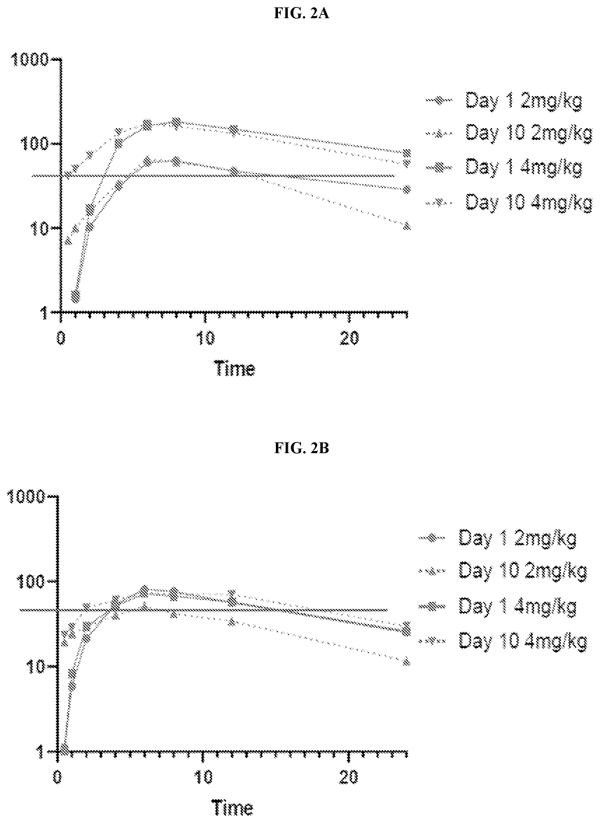

Pharmacokinetic studies of Compound (1a) in non-human primates were also undertaken in male (FIG. 2A) and female (FIG. 2B) monkeys at two different doses (2 mg/kg and 4 mg/kg) via oral administration. As shown in these figures, the curve shapes and AUC at either dose were similar on both Day 1 and Day 10 of the study, indicating no significant loss of exposure after repeat dosing.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound having a structure according to Formula (I), 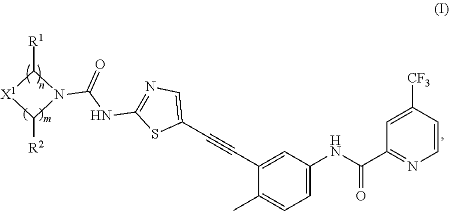
or a pharmaceutically acceptable salt, solvate, prodrug, stereoisomer, or tautomer thereof, wherein
n and m are independently 1, 2, or 3;
X¹ is O, NR³, CR^{x}R³ or CHR³;
each R¹, R², and R³ is independently H or X²;
X² is OH, O(C₁-C₆ alkyl), NH₂, NHR⁴, or NR⁴R⁵;
R⁴ and R⁵ are independently C₁-C₆ alkyl or R⁴ and R⁵, when both are present, combine to form a 4- to 6-membered heterocyclyl;
R^{x} is C₁-C₆ alkyl; and
wherein at least one of R¹, R², and R³ is X².

2. The compound of claim 1, wherein X¹ is O, NR³, or CHR³.

3. The compound of claim 1, wherein X¹ is CR^{x}R³ and R^{x} is Me.

4. The compound of claim 1, having a structure according to Formula (I′), 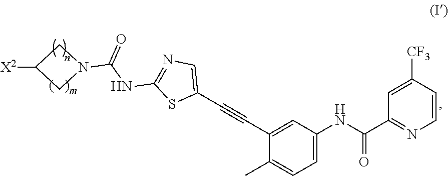
or a pharmaceutically acceptable salt, solvate, prodrug, stereoisomer, or tautomer thereof.

5. The compound of claim 4, having a structure according to Formula (I′-A), 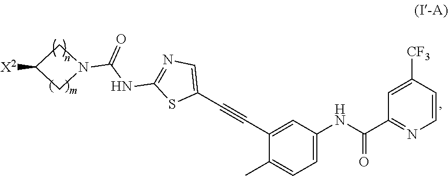
or a pharmaceutically acceptable salt, solvate, prodrug, or tautomer thereof.

6. The compound of claim 4, having a structure according to Formula (I′-B), 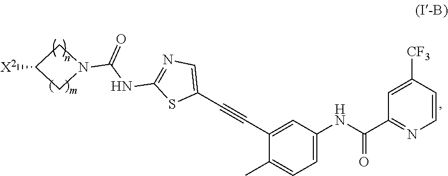
or a pharmaceutically acceptable salt, solvate, prodrug, or tautomer thereof.

7. The compound of any one of claims 1-6, wherein X² is OH, NH₂, NHCH₃, or N(CH₃)₂.

8. The compound of any one of claims 1-7, wherein X² is NH₂, NHCH₃, or N(CH₃)₂.

9. A compound having a structure selected from the group consisting of: 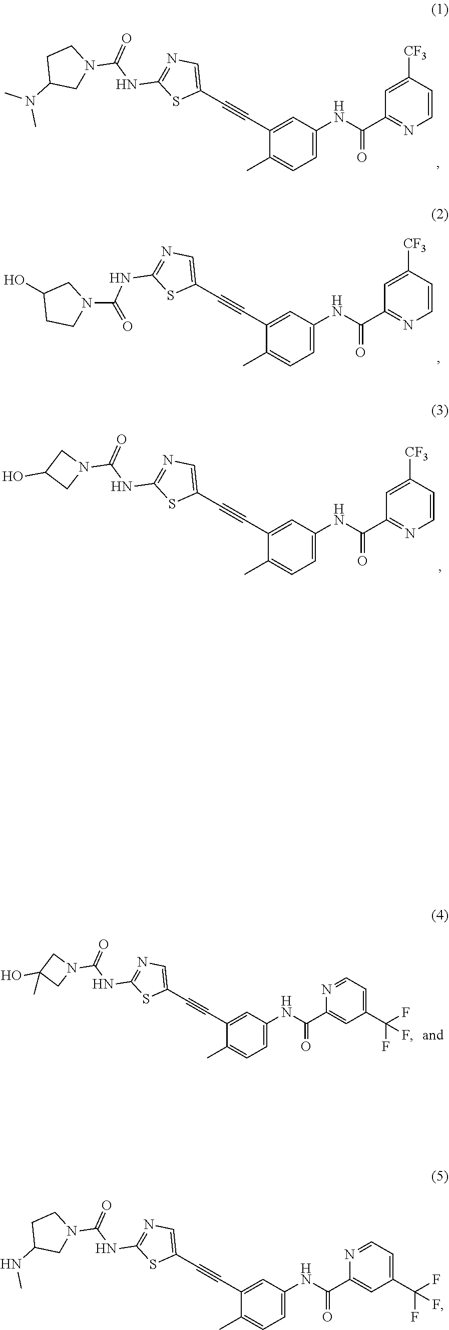 [SMILES: Cc1ccc(NC(=O)c2cc(C(F)(F)F)ccn2)cc1C#Cc1cnc(NC(=O)N2CCC(N(C)C)C2)s1 ; Cc1ccc(NC(=O)c2cc(C(F)(F)F)ccn2)cc1C#Cc1cnc(NC(=O)N2CCC(O)C2)s1 ; Cc1ccc(NC(=O)c2cc(C(F)(F)F)ccn2)cc1C#Cc1cnc(NC(=O)N2CC(O)C2)s1 ; Cc1ccc(NC(=O)c2cc(C(F)(F)F)ccn2)cc1C#Cc1cnc(NC(=O)N2CC(C)(O)C2)s1 ; CNC1CCN(C(=O)Nc2ncc(C#Cc3cc(NC(=O)c4cc(C(F)(F)F)ccn4)ccc3C)s2)C1]
or a pharmaceutically acceptable salt, solvate, prodrug, stereoisomer, or tautomer thereof.

10. The compound of claim 9, having the following structure, 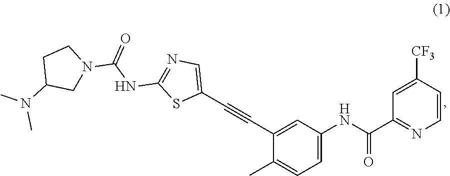 [SMILES: Cc1ccc(NC(=O)c2cc(C(F)(F)F)ccn2)cc1C#Cc1cnc(NC(=O)N2CCC(N(C)C)C2)s1]
or a pharmaceutically acceptable salt, solvate, prodrug, stereoisomer, or tautomer thereof.

11. The compound of claim 10, having the following structure, 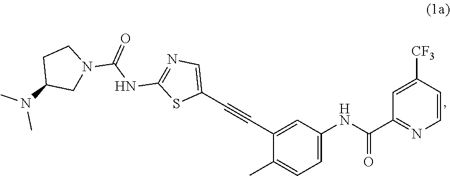 [SMILES: Cc1ccc(NC(=O)c2cc(C(F)(F)F)ccn2)cc1C#Cc1cnc(NC(=O)N2CC[C@H](N(C)C)C2)s1]
or a pharmaceutically acceptable salt, solvate, prodrug, or tautomer thereof.

12. The compound of claim 10, having the following structure, 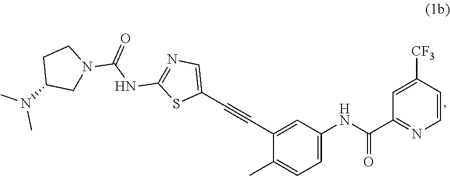 [SMILES: Cc1ccc(NC(=O)c2cc(C(F)(F)F)ccn2)cc1C#Cc1cnc(NC(=O)N2CC[C@@H](N(C)C)C2)s1]
or a pharmaceutically acceptable salt, solvate, prodrug, or tautomer thereof.

13. The compound of claim 9, having the following structure, 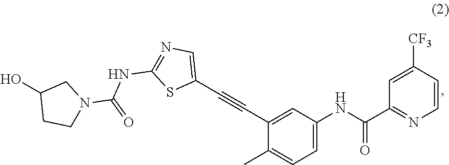 [SMILES: Cc1ccc(NC(=O)c2cc(C(F)(F)F)ccn2)cc1C#Cc1cnc(NC(=O)N2CCC(O)C2)s1]
or a pharmaceutically acceptable salt, solvate, prodrug, stereoisomer, or tautomer thereof.

14. The compound of claim 13, having the following structure, 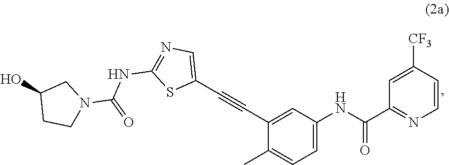 [SMILES: Cc1ccc(NC(=O)c2cc(C(F)(F)F)ccn2)cc1C#Cc1cnc(NC(=O)N2CC[C@@H](O)C2)s1]
or a pharmaceutically acceptable salt, solvate, prodrug, or tautomer thereof.

15. The compound of claim 13, having the following structure, 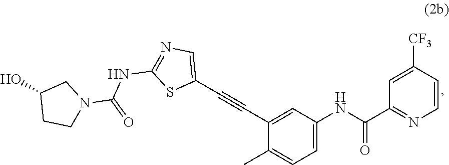 [SMILES: Cc1ccc(NC(=O)c2cc(C(F)(F)F)ccn2)cc1C#Cc1cnc(NC(=O)N2CC[C@H](O)C2)s1]
or a pharmaceutically acceptable salt, solvate, prodrug, or tautomer thereof.

16. The compound of claim 9, having the following structure, 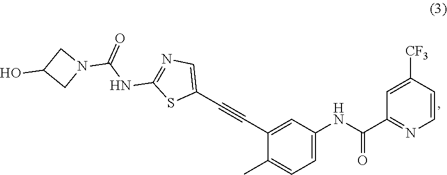 [SMILES: Cc1ccc(NC(=O)c2cc(C(F)(F)F)ccn2)cc1C#Cc1cnc(NC(=O)N2CC(O)C2)s1]
or a pharmaceutically acceptable salt, solvate, prodrug, stereoisomer, or tautomer thereof.

17. The compound of claim 9, having the following structure, 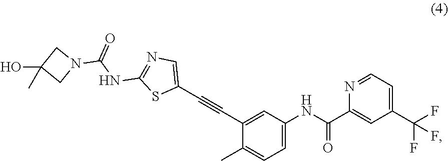 [SMILES: Cc1ccc(NC(=O)c2cc(C(F)(F)F)ccn2)cc1C#Cc1cnc(NC(=O)N2CC(C)(O)C2)s1]
or a pharmaceutically acceptable salt, solvate, prodrug, stereoisomer, or tautomer thereof.

18. The compound of claim 9, having the following structure, 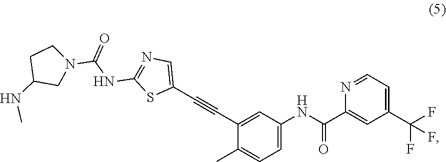 [SMILES: CNC1CCN(C(=O)Nc2ncc(C#Cc3cc(NC(=O)c4cc(C(F)(F)F)ccn4)ccc3C)s2)C1]
or a pharmaceutically acceptable salt, solvate, prodrug, stereoisomer, or tautomer thereof.

19. The compound of claim 18, having the following structure, 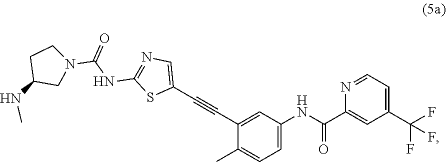 [SMILES: CN[C@H]1CCN(C(=O)Nc2ncc(C#Cc3cc(NC(=O)c4cc(C(F)(F)F)ccn4)ccc3C)s2)C1]
or a pharmaceutically acceptable salt, solvate, prodrug, or tautomer thereof.

20. The compound of claim 18, having the following structure, 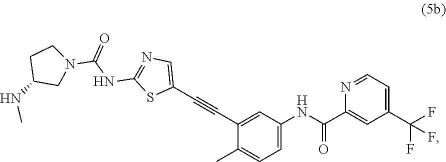 [SMILES: CN[C@@H]1CCN(C(=O)Nc2ncc(C#Cc3cc(NC(=O)c4cc(C(F)(F)F)ccn4)ccc3C)s2)C1]
or a pharmaceutically acceptable salt, solvate, prodrug, or tautomer thereof.

21. A pharmaceutical composition comprising a compound of any one of claims 1 to 20, or a pharmaceutically acceptable salt, solvate, prodrug, stereoisomer, or tautomer thereof, and a pharmaceutically acceptable diluent, excipient or carrier.

22. A method of treating a c-Kit-mediated disease or disorder, comprising administering to a subject in need thereof a therapeutically effective amount of a compound of any one of claims 1 to 20, or a pharmaceutically acceptable salt, solvate, prodrug, stereoisomer, or tautomer thereof.

23. The method of claim 22, wherein the cKit-mediated disease or disorder is selected from cell proliferative disorder, a fibrotic disorder, and a metabolic disorder.

24. The method of claim 23, wherein said cell proliferative disorder is cancer.

25. The method of claim 24, wherein said cancer is selected from the group consisting of leukemia, mast cell tumor, small cell lung cancer, testicular cancer, cancer of the gastrointestinal tract, cancer of the central nervous system, cancer of the female genital tract, sarcoma of neuroectodermal origin, and Schwann cell neoplasia associated with neurofibromatosis.

26. The method of claim 24, wherein said cancer is selected from the group consisting of small cell lung carcinoma, acute myeloid leukemia (AML), neuroblastoma, malignant melanomas, colorectal cancer, systemic mastocytosis (SM), and gastrointestinal stromal tumors (GISTs).

27. The method of claim 22, wherein said cKit-mediated disease or disorder is multiple sclerosis.

28. The method of claim 22, wherein said cKit-mediated disease or disorder is asthma.

29. The method of claim 22, wherein said cKit-mediated disease or disorder is an allergic reaction.

30. The method of claim 22, wherein said cKit-mediated disease or disorder is inflammatory arthritis.

31. The method of claim 22, wherein said cKit-mediated disease or disorder is mastocytosis.

32. A method of modulating c-Kit comprising administering to a subject a compound of any one of claims 1 to 20.

33. A method of inhibiting c-Kit comprising administering to a subject a compound of any one of claims 1 to 20.

34. A method of treating or preventing a disease in which c-Kit plays a role, comprising administering to a subject in need thereof an effective amount of a compound of any one of claims 1 to 20.
